(19) United States
(12) Patent Application Publication
(10) Pub. No.: US 2010/0106036 A1
(43) Pub. Date: Apr. 29, 2010

(54) ARRYTHMIA ADJUDICATION AND THERAPY TRAINING SYSTEMS AND METHODS

(75) Inventors: Yanting Dong, Shoreview, MN (US); Scott A. Meyer, Lakeville, MN (US); Deepa Mahajan, Minneapolis, MN (US); Haresh G. Sachanandani, Culver City, CA (US); Kenneth P. Hoyme, Plymouth, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 12/582,460

(22) Filed: Oct. 20, 2009

Related U.S. Application Data
(60) Provisional application No. 61/108,674, filed on Oct. 27, 2008.

(51) Int. Cl. A61B 5/0402; G09B 23/28

(52) U.S. Cl. 600/523; 434/262

(57) ABSTRACT

A system and method for presenting arrhythmia episode information to a user are described. An episode database has episode data regarding a plurality of different arrhythmia episodes generated from a plurality of data-generating devices. A user interface is configured to display the episode data for one of the arrhythmia episodes and receive characterization data from the user characterizing the displayed episode data. An adjudication database has adjudication conclusions associated with the arrhythmia episodes in the episode database. The system further includes an adjudication processor, configured to process characterization data relative to the adjudication database.

This application claims the benefit of U.S. Provisional Application No. 61/108,674, filed Oct. 27, 2008, the content of which is herein incorporated by reference in its entirety.

### TECHNICAL FIELD

This disclosure relates generally to medical systems and, more particularly, to medical systems that can be used to communicate with implanted medical devices, amongst other things.

### SUMMARY OF THE INVENTION

Embodiments of the invention are related to medical systems and methods that can be used to communicate with and collect information from implanted medical devices, amongst other things.

In one embodiment, a system for presenting arrhythmia episode information to a user includes an episode database having episode data regarding a plurality of different arrhythmia episodes generated from a plurality of data-generating devices. The system further includes a user interface configured to display at least a particular portion of episode data for one of the arrhythmia episodes from the episode database and receive characterization data from the user characterizing the displayed episode data. The system also includes an adjudication database having adjudication conclusions associated with the arrhythmia episodes in the episode database. The system further includes an adjudication processor, configured to process characterization data relative to the adjudication database.

In another embodiment, a method includes the step of graphically displaying a portion of data selected from arrhythmia episode data related to an arrhythmia episode of a patient, wherein the arrhythmia episode data was generated by a data-generating device. The method further includes requesting user input characterizing the episode, receiving the characterization data from the user, assessing similarities of characterization data relative to an adjudication decision, and reporting similarities of user input relative to the adjudication decision.

In yet another embodiment, a method of developing a training database for training users to identify arrhythmias includes the steps of graphically displaying a portion of data selected from arrhythmia episode data, requesting user input characterizing the event, assessing similarities of user input relative to an adjudication decision in an adjudication database, and reporting similarities of user input relative to the adjudication decision. In addition, if the user is designated as an expert, the step of recording the user input in the adjudication database is performed.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a schematic illustration of one example of a training content request user interface screen that can be used to request viewing of specific types of episode data.

FIG. 2 is a schematic illustration of another example of a training content request user interface screen that can be used to request viewing of specific types of episode data.

FIG. 3 is a schematic illustration of a user interface screen in accordance with various embodiments herein for training on arrhythmia classification.

FIG. 4 is a schematic illustration of a user interface screen in accordance with various embodiments herein for training on therapy options.

FIG. 5 is a schematic illustration of another user interface screen in accordance with various embodiments herein for training on therapy options.

FIG. 6 is a schematic illustration of an embodiment of a user interface screen for presenting training success rate results to a user.

FIG. 7 is a flowchart showing one embodiment of a method of training

FIG. 8 is a flowchart showing another embodiment of a method of training FIG. 9 is a schematic diagram of an exemplary implementation of a cardiac rhythm management (CRM) system, including an implanted CRM device, an external interface device, and a patient management computer system, consistent with at least one embodiment of the invention.

FIG. 10 is a schematic illustration of a patient management system consistent with at least one embodiment of the invention.

FIG. 11 is a schematic diagram of an implementation of the components of an external interface device such as a programmer, in accordance with various embodiments.

FIG. 12 is a schematic view of components of an implantable medical system in accordance with an embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

This disclosure relates generally to medical data-generating devices and, more particularly, to systems that collect information from sensor readings provided by medical devices. In particular, this disclosure relates to systems and methods for providing training on how to classify diagnose or treat patients based on the use of the information collected from the medical devices. In some embodiments, the medical devices are implanted. In some embodiments, this disclosure also relates to providing training on how to program the implanted medical devices. Still more specifically, the disclosure is especially relevant in the context of medical devices that monitor cardiac rhythms, including but not limited to cardiac rhythm management systems (CRM systems) that include an implantable cardiac rhythm management device (CRM device), an external interface device and a patient management computer system.

Some implantable medical devices can be used to provide pacing therapy to patients who have cardiac rhythm problems. For example, an implanted cardiac rhythm management (CRM) device can be used to provide pacing therapy to a patient with sinus node dysfunction, where the heart fails to properly initiate depolarization waves, or an atrio-ventricular conduction disturbance, where the conduction of depolarization waves through the heart tissue is impaired.

Many types of CRM devices communicate with devices located outside of the body, which can receive information from the implanted device including sensor information and information about events, such as when the implanted device has provided therapy. In some cases, the external interface device can also transmit operational parameters to an implanted CRM device, that is, program the device.

These external interface devices are often provided to a patient, often in a patient's home, and can collect information from the implanted device, and provide that information to a computer system designed to monitor the patient's status. An exemplary in-home monitoring system is the LATITUDE® patient management system, available from Boston Scientific Corporation, Natick, Mass. Aspects of exemplary monitoring systems are described in U.S. Pat. No. 6,978,182, the content of which is herein incorporated by reference in its entirety.

The existence of in-home monitoring systems such as the LATITUDE® patient management system has provided a large amount of data about patients with implanted medical devices. For example, these systems store information about patient characteristics, patient sensor readings including electrocardiogram (EGM), device settings and delivery of therapy by the device. The sensor readings can include information associated with arrhythmia episodes experienced by the patient.

In one embodiment, a training system uses a storehouse of patient-related data for training users with regard to classifying and diagnosing patient episodes and programming medical devices in response to an episode. “Episode” is defined to mean activity of a patient's body within a time period of particular interest. The time period can be a time when there is abnormal activity, for example, abnormal cardiac activity. Episode data includes sensor readings from a medical device during the episode, and can also include device settings, actions that were taken by the device and other information as detailed herein. An episode database stores the episode data about episodes that have occurred.

For a particular episode, the episode data or part of the episode data can be displayed to a user on a user interface. The user is asked to characterize the episode data, also via the user interface. An adjudication processor processes the characterization data based on adjudication conclusions that are stored in an adjudication database. The adjudication processor may determine that the characterization data is correct, is incorrect, or determine a percentage of agreement between the characterization data and the adjudication conclusions. The level of agreement is then reported to the user.

So far, the example of implantable cardiac rhythm management devices has been described for generating the episode data. Specific implantable cardiac rhythm management devices include a pacemaker, a cardioverter-defibrillator, a cardiac resynchronization device, a heart rhythm monitoring device, or the like. However, it is also possible to generate episode data from external devices, including external pacemakers, external cardioverter-defibrillators, and external resynchronization devices. Additional examples of external devices that monitor cardiac activity include ambulatory electrocardiography devices or Holter monitors, which continuously monitor electrical activity of the heart for 24 hours or more.

In various embodiments, users have identities recognized by the system and are authenticated before training begins. Authentication can comprise entering in a user identity, such as a handle, identification number, or the like, and entering a corresponding password in a user interface. Other methods of authentication can also be employed.

The system is capable of storing user information and storing the success rate of the user. The success rate can be determined based on a comparison of the characterization data entered by the user to the adjudication conclusions. Such success rate can be determined over the course of a user's training history. In one embodiment, if a user has a success rate over a threshold success rate, the user is classified as an expert. The effect of expert designation is further described herein.

In one embodiment, adjudication conclusions are determined by a panel of knowledgeable individuals who have reviewed the episode data. The adjudication conclusions may be the conclusions of at least one member of the panel, or may require agreement among two or more members of the panel. In yet another embodiment, adjudication conclusions are determined and entered into a database by an adjudication processor based on the characterization data entered by all users. In such an embodiment, the characterization data entered by users classified as experts may be given more weight than other users. Alternatively, only characterization data entered by users classified as experts is entered into the adjudication database. In one embodiment, the adjudication conclusions are panel decisions where panel decisions are available, are based on expert-level user classifications where available, and are based on characterization data entered by all users where available.

The system can also provide a training content request that allows the user to indicate what the type of training and/or what types of episodes will be used for the training. The training content request can also, in various embodiments, be entered automatically by the system based on the user identity. In other embodiments the training content request can be entered by a third party, such as an instructor or supervising doctor, for example. It is possible that the training content request requires multiple inputs that are received from various sources. In one example, the training content request allows input as to one or more of the following criteria: patient demographics, patient identity, device type, lead types, patient disease status, patient medical history, a device-generated arrhythmia classification, level of difficulty, and operating mode. The training content request may also specify whether the user will be tested on classifying cardiac arrhythmia episodes or programming device settings.

In an example system particular to implanted cardiac rhythm management systems, training content could be specific to device types such as a single chamber implantable cardioverter defibrillator (ICD), dual chamber ICD, cardiac resynchronization therapy defibrillator (CRT-D), or any device type. The user can ask to be presented with arrhythmia episodes that the device has classified as ventricular tachycardia, ventricular fibrillation, supraventricular tachycardia or any arrhythmia type.

Training content requests can also allow the user to specify one or more device settings that were present during the episode, such as whether detection enhancements were turned on or off, whether morphology-based discrimination was turned on or off, and whether therapy programming was to be shock energy, anti-tachycardia pacing (ATP) only, or any therapy programming.

It is also possible for the system to be configured to allow the user to specify the type of episode that will be viewed by selecting arrhythmic parameters, which are sensor readings or determinations that the device makes based on sensor readings during an arrhythmia episode. Examples of arrhythmic parameters include the patient's atrial rate, the patient's ventricle rate, the morphology type, the morphology correlation rate, the rate stability, the onset, and the mode switch status. Use of arrhythmic parameters, especially in combination with patient demographic criteria, allows the user to winnow down to a set of similar episodes, which can be helpful during the training process.

Training content requests can also specify different levels of difficulty of classification of the particular episode, such as easy and difficult, or easy, normal, and difficult, although many other labels for difficulty levels could be used. The difficulty level of a particular episode can be determined by a panel of experts or by expert users. Also, an episode may be declared difficult by the system where many different arrhythmia classifications have been received from different users. An episode may be declared easy by the system if users are consistent with entering a specific arrhythmia classification. Also, arrhythmia episodes can be selected as being easy, difficult or interesting as classified by other users.

Training content could be determined based on operating the system in different modes, such as a beginner and expert mode. Depending on the degree of difficulty indicated, a user interface can be configured to display episodes outside of the content criteria to test the user's ability to discern differences.

The episode database includes episode data from all the patients participating in a patient management system in one embodiment. In an embodiment, the episode data provided to the user includes arrhythmia episode data from a patient group which have authorized the user to view the arrhythmia episode data. For example, episode data from patients within a specific clinic could be presented. In selecting which arrhythmia episodes to present to a user, the system in one embodiment will give a priority to episodes from patients with more than a certain number of episodes stored in the episode database. Also, the user can elect to be provided with episode data from a single patient or subset of patients.

Examples of patient demographic criteria that can be selected by the user in some embodiments include the patient's gender, the patient's age, the patient's weight, and the patient's disease status such as if they have diabetes. A patient's region, country or city of residence can also be used in one embodiment.

A user interface is used to select the features of the training content request. The user may check boxes offering one, two, three or more of the choices described above. The user may also use a text field to type in the desired criteria.

The user or third party will also select the type of training that they would like the user to receive in the training content request form. In one embodiment, a choice is offered between arrhythmia adjudication training and therapy training. In one embodiment of arrhythmia adjudication training, the user is shown episode data related to an arrhythmia episode, and the user is prompted to characterize the type of arrhythmia that is present in the particular arrhythmia episode. In one embodiment of therapy training, the user is also provided with most of the episode data but is not shown the type of therapy that was provided by the device. The user selects therapy options that would be appropriate for treating the arrhythmia episode. More detail regarding various arrhythmia adjudication and therapy training embodiments will be described herein.

After the submission of the training content request, the system selects appropriate arrhythmia episodes for presentation to the user. Episode data can be stored in an episode database. The episode data can be presented via a visual display using a combination of graphs, text and other images. The episode data can include a statement of the device type which generated the data, such as a Holter monitor, single chamber implantable cardioverter defibrillator (ICD), dual chamber ICD, cardiac resynchronization therapy defibrillator (CRT-D) or other devices. The episode data can also include electrocardiogram (EGM) recorded during the duration of the episode. The EGM may be generated by an implanted device. The EGM is an intracardiac EGM in one embodiment. In one embodiment, a portion of the EGM is displayed, and the user has the ability to scroll, click on fields, or otherwise interact with the user interface to see other parts of the EGM. The EGM may include a display of electrical pulses sensed in the atrium and ventricle portions of the heart, as well as a display of the pacing pulses generated by the device.

The episode data can also include a device-generated arrhythmia classification such as non-sustained ventricular tachy arrhythmia (VT), sustained ventricular arrhythmia, atrial fibrillation (AF) and supraventricular tachycardia (SVT). In one embodiment, the episode data also includes pace and sense markers on the EGM, which can indicate when along the course of the EGM the patient's heart rate can be classified in certain ways, such as ventricular tachy arrhythmia (VT), atrial fibrillation (AF) and supraventricular tachycardia (SVT).

In one embodiment, the episode data also includes device diagnostics which are measurements recorded by the device, and which can include average atrial heart rate, average ventricular heart rate and many other measurements.

The episode data can also include the device's response to the arrhythmia, such as anti-tachycardia pacing (ATP) therapy or shock therapy, and more specific descriptions of the therapy. The response information can be shown on the EGM strip and/or textually. In certain embodiments, the episode data can further include information on how the patient responded to the therapy, and information on subsequent therapy that was delivered.

In addition, the episode data can also include the device settings that were present during the episode. Examples of device settings include therapy settings such as what type of pacing or shock therapy will be applied when the device concludes that certain cardiac arrhythmias are occurring. For example, ATP therapy involves delivering a series of timed pacing pulses where the user can define the number of bursts delivered, the number of pacing pulses within each burst, and many other criteria. ATP therapy can fall into several different schemes, and each device has its own terminology describing the ATP therapy schemes. For some devices available from Boston Scientific Corporation, some of the ATP therapy schemes are named a burst scheme, a ramp scheme, and scan scheme or a ramp/scan scheme. The specifics of these ATP therapy scheme names, and when they will be delivered to the patient, are examples of device settings that may be viewable by the user as a part of the episode data.

Another type of device setting is shock therapy parameters, which govern the shock energy and timing of shock delivery, as well as many other qualities of shock therapy.

Other types of device setting include detection settings that set forth the heart rate, rate of onset, and other criteria by which the device will classify the episode as certain arrhythmia types. Another type of device setting is frequently referred to as detection enhancements. These settings can be used to ensure that it is appropriate to delivery therapy to the patient by checking for the presence or absence of certain criteria. If the criteria are present, the detection enhancement settings may cause the device to delay or inhibit therapy delivery, bypass therapy inhibition, or bypass a sequence of ATP therapy in favor of shock therapy. Examples of specific detection enhancement criteria include the following, where the name for the programming feature for some devices available from Boston Scientific Corporation is included in parenthesis: certain therapy inhibitor conditions can be bypassed if the ventricular rate is greater than the atrial rate (V Rate>A Rate), ventricular therapy can be inhibited if the atrial rhythm is too fast (AFib Rate Threshold), ventricular therapy can be inhibited if the ventricular rhythm is unstable (Stability), and ventricular therapy can be inhibited if the patient's heart rate increases gradually (Onset). These parameters and whether they are turned on or off can be display along with other device settings in one embodiment.

Many other device settings are available for certain devices. Some of these additional device settings are a part of the available episode data in some embodiments.

The episodes may be episodes stored for atrial arrhythmia therapy.

After presenting the episode data, the system requests user input characterizing the event, and the user enters in characterization data about the episode, which characterizes the episode data in some way.

In arrhythmia adjudication training embodiments, the requested characterization data is an arrhythmia classification of any arrhythmia that is present during the episode. Examples of arrhythmia classifications that may be entered in some embodiments are polymorphic ventricular tachycardia (PVT), ventricular fibrillation (VF), monomorphic ventricular tachycardia (MVT), supraventricular tachycardia (SVT), AV nodal re-entrant tachycardia (AVNRT), atrial fibrillation (AF), atrial flutter (AFL), atrial tachy, sinus tachy (ST), dual tachy, indeterminate, oversensing and noise. Another possible arrhythmia classification is pacemaker-mediated tachy (PMT). The user may select one of these classifications from a list, or may enter the information into a text field.

In one embodiment, the user is prompted to indicate which part of the episode data is the main basis for the user's arrhythmia classification. In one example, the user can mark a portion of the EGM as forming the basis for their conclusion. The user can also enter comments about the episode in one embodiment. In addition, the user can mark the episode as fitting into a certain category in the database, such as interesting, easy or difficult, in one embodiment. Where a user or a threshold number of users have marked an episode as interesting, that episode is provided to a panel for review, in one embodiment. The user can also request panel review of a specific arrhythmia episode.

In one embodiment, the user is asked to provide a rationale for the arrhythmia classification. In some embodiments, the rationale is provided entirely by the user in a text field. However, in some embodiments, the rationale is selected by the user from a pre-defined list. For example, if the episode is characterized as a ventricular tachycardia than the predefined rationale options could be:

(1) V rate>A rate
(2) morphology different from sinus rhythm
(3) stable ventricular rhythm
(4) tachy initiated by V. and
(5) other.
If the episode is characterized as an SVT then the predefined rationale options could be:
(1) A rate>V rate,
(2) A associated one to one with V
(3) unstable ventricular rhythm
(4) Tachy initiated by A, and
(5) other.

For arrhythmia adjudication training screens, the user is not presented with any of the device setting information in one embodiment. In one embodiment, the user is presented with the following data related to the episode: the EGM, the device type, and the device-generated arrhythmia classification. In another embodiment, the user is also provided with pace and sense markers, intervals and device diagnostics or measurements. In another embodiment, the user is also presented with information regarding the therapy delivered to the patient.

There are a variety of system embodiments that can be employed for therapy training which can be referred to as programming training. In one embodiment, a user is kept unaware of the therapy that was provided by the device, because that portion of the episode data is not presented to the user. Also, the device settings are not presented to the user. Based on the user's knowledge of cardiac arrhythmias, the user characterizes the episode event in terms of selecting corresponding therapy. In these therapy training embodiments, the therapy and device settings are the characterization data that is requested of the user.

As discussed above, there are many therapy options and device settings that are available. A subset of these can be presented to the user for allowing the user to enter parameter selections. In one embodiment, the system presents a list of detection enhancements and the user selects whether each enhancement should be turned on or off. In another embodiment, the system presents morphology-based discrimination settings, and the user is prompted to select which are turned on or off. In another embodiment, specific therapy programming settings such as shock energy or ATP parameters are presented to the user for entry of values.

In another embodiment, tachycardia detection parameters, including the number of zones, rate cutoffs for each zone, initial detections durations, and sustained rate duration setting, are to be entered by the user in response to the episode data.

In one embodiment, the user is prompted to indicate which part of the episode data is the main basis for device settings. For example, the user could note that the atrial, ventricle, shock or a combination of the parts of the EGM was the basis for the device settings. Such a designation could be helpful in the review of difficult episodes.

The user characterization is then compared with the adjudication conclusion, which was arrived at through a panel selection or a selection by other users. “Real-world” performance or response of the device using the particular user-selected characterization data could potentially be provided to the user to educate the user. In another embodiment a user can be kept unaware of a particular data point collected by the data-generating device, and be provided the opportunity to characterize what that particular data point may be. In such a scenario the adjudication data for the episode is provided by the data-generating device.

The adjudication conclusion includes some or all of the same aspects as characterization data entered by the user, in some embodiments, such as a rationale for the selection of a particular arrhythmia classification and the other types of characterization data described herein.

The system then assesses the similarities of the user input characterizing the data to an adjudication decision in an adjudication database and may report on the similarities. The user input can also be recorded in the adjudication database in corresponding embodiments. The user may be given the opportunity to compare the characterization data entered to the adjudication decision. This comparison can be helpful particularly when there is not a consensus as to the adjudication decision, such as when adjudication decisions are made by a panel of experts or user-entered characterization data. The comparison can also help the user see that there may be agreement with a certain aspect of the rationale even if there is not agreement with the arrhythmia classification. A summary of the results can also be presented, such as the percentage of episodes correctly characterized, or a measurement of the agreement of characterization data with experts, other users, or a panel.

After entering characterization data, in one embodiment the user is able to view the characterization data entered by other users, possibly by viewing overall trends in characterization data. Episodes with diverse characterization data can be candidates for a panel review to refine and solidify the adjudication data. In some embodiments a user may submit a particularly episode for panel review.

As described above, an adjudication database can be populated with adjudication conclusions based on conclusions from a variety of sources. Each adjudication conclusion corresponds to a particular episode from a particular data-generating device. A group of episodes are selected to be adjudicated by a panel, and in a variety of embodiments the panel is composed of physicians. The episode database is generally updated regularly to include new episodes in the system and refine and solidify the adjudication data.

As mentioned above, where user characterization data is entered into the adjudication database, the system can incorporate a mechanism to attract more users, especially those who are particularly knowledgeable. Such a mechanism may be set up in the form of challenging users to correctly characterize a certain percentage of episodes, where “correctly characterizing” means that the characterization data sufficiently matches the adjudication conclusion according to the requirements of the system, the particular episode data, and/or the panel. Users who meet the challenge can be referred to as “experts”. Experts may be ranked at a base level compared to other users, for example, the top 10% of users, and may also be further ranked, for example, the top 5% of users. Users can be informed of their achievements by the system, which will motivate some users to higher use rates. The characterization data entered by experts are generally given more weight in the adjudication database then characterization data entered by users who are not classified as an expert. An alternative mechanism may recognize those users who characterize the most number of episodes.

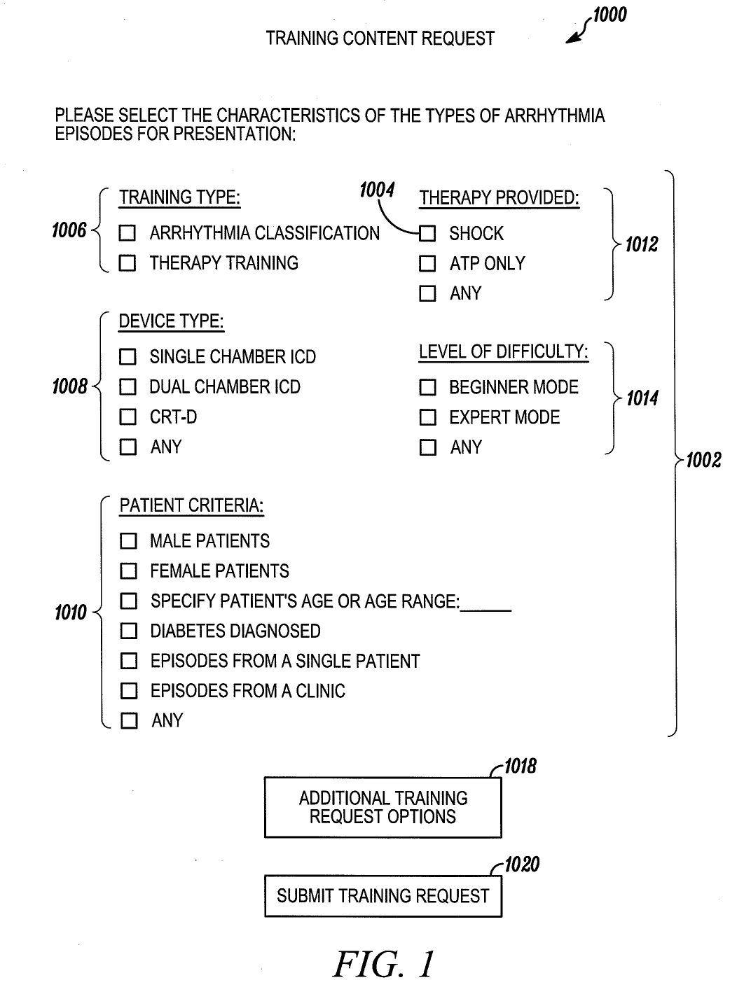

Further detailed embodiments and user interface screens examples will now be described with respect to the attached FIGS. FIG. 1 is a schematic illustration of one example of a training content request user interface screen 1000 that can be used to request viewing of specific types of episode data. The user is presented with a range of options 1002, as further described herein in detail, for selecting qualities of the arrhythmia episodes. Checkboxes 1004 are provided in one embodiment near each of the options for selection.

In one embodiment, training type options 1006 including arrhythmia classification and therapy training are presented to the user. Device type options 1008 such a single chamber ICD, dual chamber ICD, CRT-D, and any device type can be selected using the checkboxes in the embodiment of FIG. 1. Patient criteria options 1010 allow the user to specify a patient's gender, age or age range, and disease diagnosis such as diabetes. The user can also choose episodes from a single patient or from the patients of a clinic. The user is also allowed to select which therapy was provided during the episodes, using the therapy options 1012. The level of difficulty options 1014 such as beginner mode, expert mode or both, can also be used to cater the training session. Once the user has made selections from all of the options 1002, the user can use additional options button 1018 to view more training content request options. Alternatively, the user can submit the training request using submit button 1020.

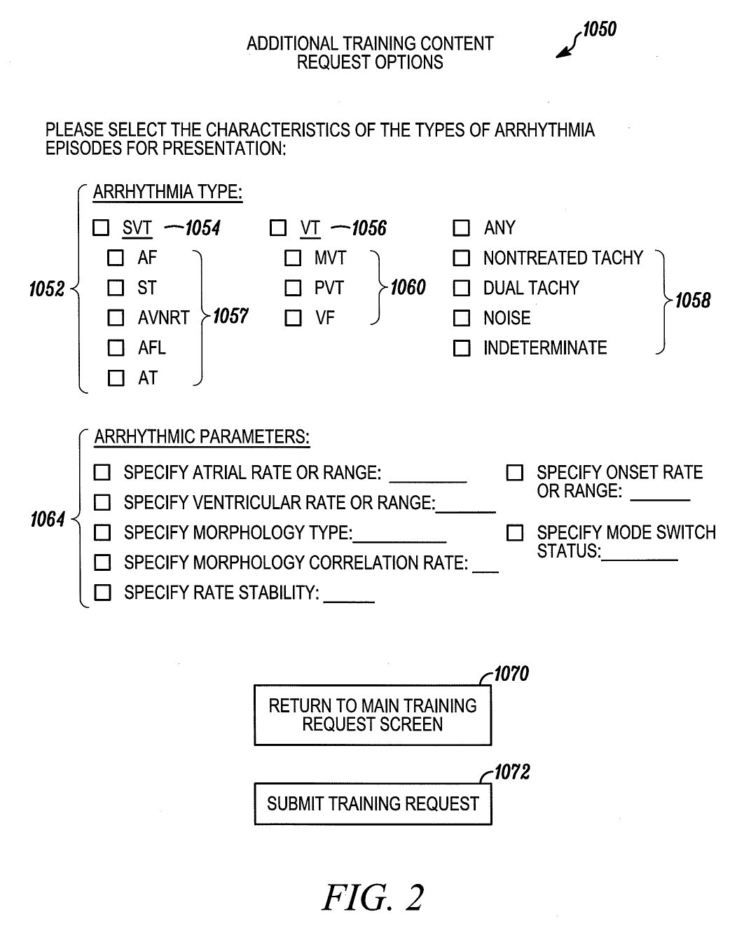

In one example, the additional options button 1018 of FIG. 1 will cause the user to be presented with screen 1050 shown in FIG. 2. The arrhythmia type options 1052 allow the user to specify which type of arrhythmia is present in the episodes that are presented to the user. For example, the user could select one or more of the general arrhythmia types such as the SVT option 1054, VT option 1056, or other options 1058 that are typically determined by the device, such as where the device determines that the arrhythmia is nontreated tachy, dual tachy, noise or indeterminate. The user could also select one of the more specific options listed in section 1057 or 1060 of the screen, where these more specific options would be identified by other a panel of experts or by other users of the system.

Arrhythmic parameter options 1064 allow the user to specify the type of episode that will be viewed by selecting arrhythmic parameters, such as the patient's atrial rate, the patient's ventricle rate, the morphology type, the morphology correlation rate, the rate stability, the onset, and the mode switch status. Use of arrhythmic parameter options 1064, especially in combination with patient criteria options 1010, allows the user to winnow down to a set of similar episodes, which can be helpful during the training process.

Once the user has made selections from all of the options presented on screen 1050 of FIG. 2, the user can use the return button 1070 to return to the main training content request screen. Alternatively, the user can submit the training request using submit button 1072.

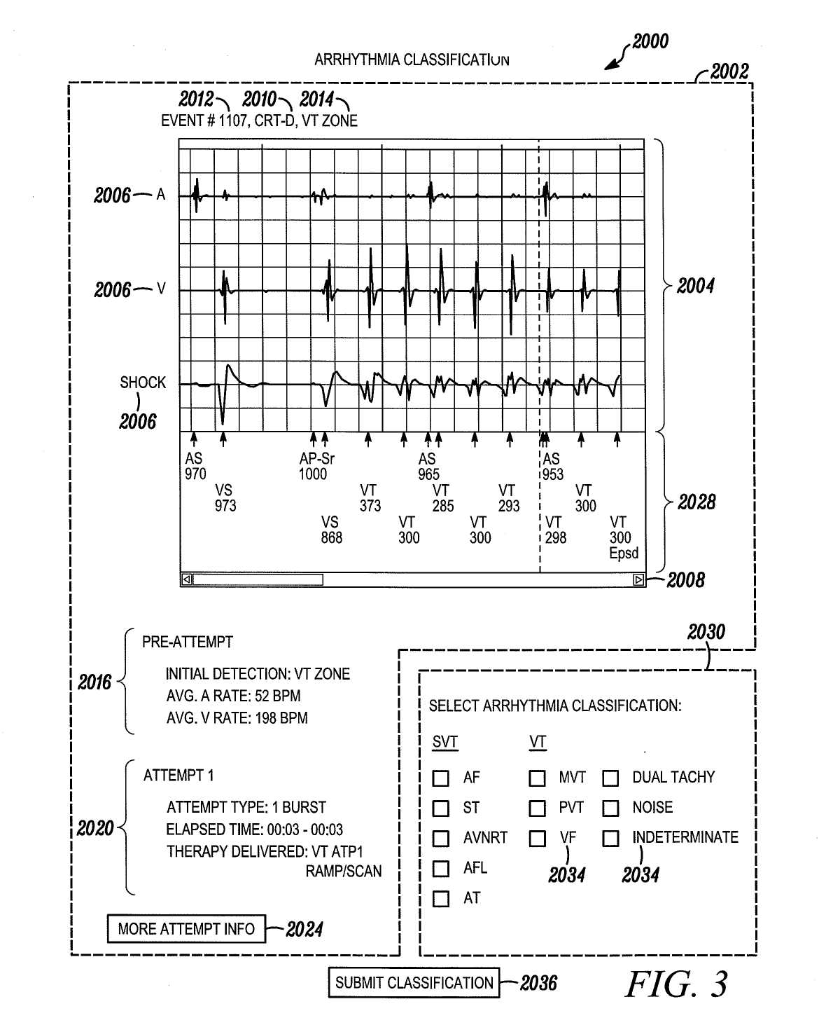

FIG. 3 is a schematic illustration of a user interface screen 2000 in accordance with various embodiments herein for training on arrhythmia classification. The user interface screen 2000 displays episode data 2002 that includes an electrocardiogram (EGM) 2004. In this embodiment the EGM is accompanied by labels 2006 stating which parts of the EGM are sensed in the atrial and ventricular portions of the heart, as well as a label for the pacing pulses generated by the device. A scroll bar 2008 is also provided in this example to allow the user to view other parts of the EGM associated with the episode.

A statement of the device type 2010 is also present, indicating in this example that the device is a CRT-D. The screen also includes an episode number 2012 for identifying the episode within the episode database. The screen also includes a label 2014 of how the device has classified the episode, which in this case indicates ventricular tachy arrhythmia (VT).

The screen 2000 further displays device diagnostic information 2016, including average heart rates and the zone into which the device has classified the arrhythmia and therapy information 2020. It is also possible for the user to activate a link or button 2024 to obtain further attempt and diagnostic information about the episode in the embodiment of FIG. 3.

Pace and sense markers 2028 are also present in the example screen shot of FIG. 3, located below the EGM, to indicate ventricular sensing (VS), atrial sensing (AS) and the detection of ventricular tachy arrhythmia (VT).

The user interface screen 2000 further displays a user input request 2030 that includes characterization options 2034. The user responds by entering in characterization data into the user interface, with one of the presented options such as polymorphic ventricular tachycardia (PVT), ventricular fibrillation (VF), monomorphic ventricular tachycardia (MVT), supraventricular tachycardia (SVT), AV nodal re-entrant tachycardia (AVNRT), atrial fibrillation (AF), atrial flutter (AFL), atrial tachy, sinus tachy (ST), dual tachy, indeterminate, PMT, oversensing and noise.

When the user is satisfied with the selected answer, the user can select a field 2036 that will initiate a checking of the user characterization against the adjudication database.

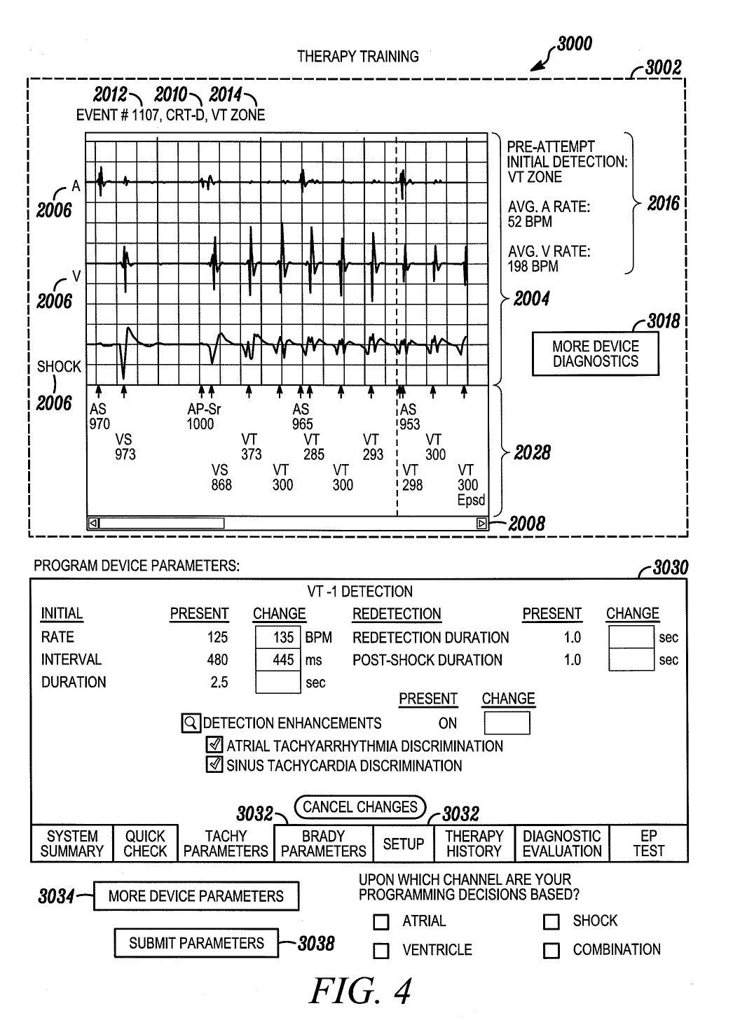

FIG. 4 is a user interface screen for therapy training where the provided with a portion of episode data related to an arrhythmia episode, and the user is prompted to enter appropriate device parameters and settings. Like in the arrhythmia adjudication embodiment of FIG. 3, the user interface screen 3000 displays episode data 3002 that includes an electrocardiogram (EGM) 2004, accompanied by labels 2006 and a scroll bar 2008. A label of the device type 2010 is also present, indicating in this example that the device is a CRT-D. The screen also includes an episode number 2012 for identifying the episode within the episode database. The screen also shows in which detection zone the episode was detected by the device at label 2014, which in this case indicates that the episode was detected in the VT zone.

The screen 3000 further displays device diagnostic information 2016, and a link 3018 for the user to obtain additional device diagnostic information. However, in contrast to FIG. 3, the screen does not display therapy settings. Pace and sense markers 2028 are also present in the example screen shot of FIG. 4.

The therapy training user interface screen 3000 further displays a user input request 3030 that prompts the user to enter device parameters. In FIG. 4, the fields for one zone of ventricular tachycardia (VT-1) are shown and are available for the user to enter appropriate parameters. Each field may have a menu of options, or may allow the user to enter text. Tabs 3032 are provided for entering other device parameters.

As shown at the bottom right corner of screen 3000, the user can be prompted to enter a response as to which channel is the basis for the programming decisions: atrial, ventricle, shock or a combination.

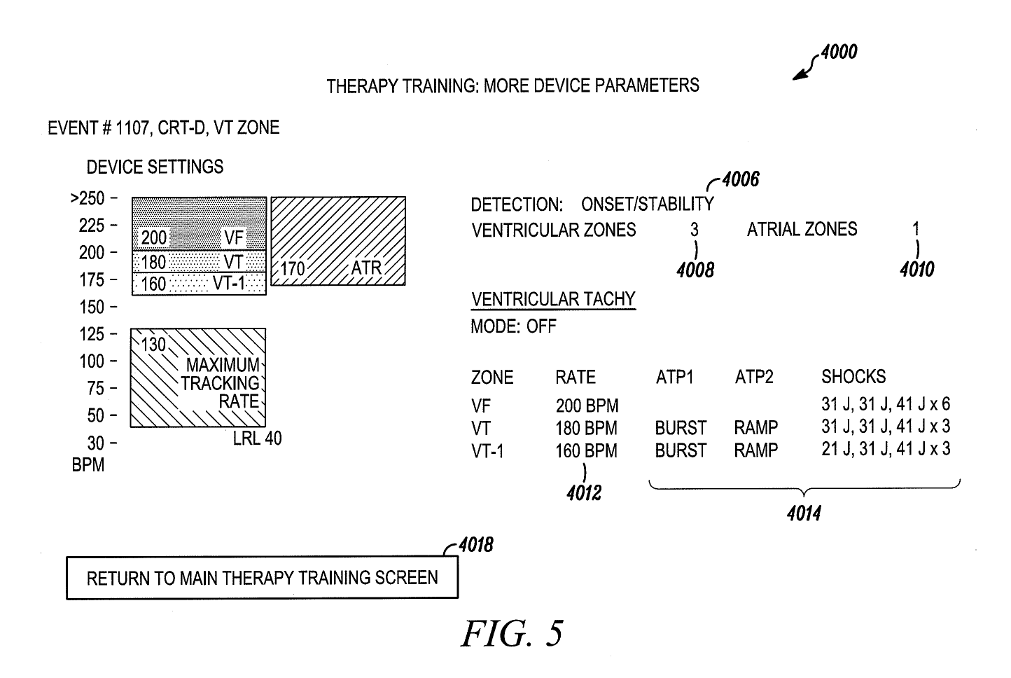

A link 3038 is provided for submitting the entered parameters to be compared to the adjudication database. A link 3034 is also provided for entering still further device parameters. In one embodiment, the link 3034 will cause the screen 4000 of FIG. 5 to be presented to the user. At the screen 4000, the user is prompted to enter other device parameters including detection parameters for ventricular tachycardia. Requested parameters include a detection enhancement setting 4006, a number of ventricular zones 4008, a number of atrial zones 4010, rate thresholds for each ventricular zone 4012, and therapy settings for each ventricular zone 4014. When the user has completed the entries available on screen 4000, a return button 4018 may be used to return to the main therapy training screen.

As discussed elsewhere herein, there are many different therapy parameters that can be requested of the user. In various embodiments, fewer therapy parameters than displayed in FIGS. 4 and 5 are to be entered by the user. Particularly in the context of embodiments of the screen shot used with a beginner-level user, fewer device parameters could be presented for selection.

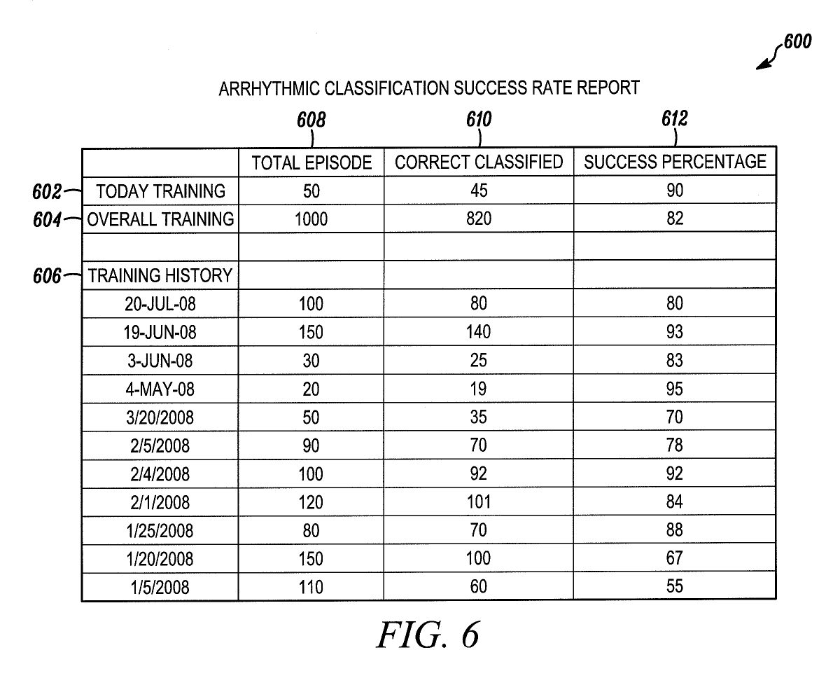

FIG. 6 illustrates an example of a success rate report 600 that may be provided to a user on an interface screen after completing an arrhythmia adjudication training session. In this example, the row labeled “Today Training” 602 includes information about the current day of training, while the row titled “Overall Training” 604 provides information about all of the user's past training Below the “Training History” row 606, entries are present for days where training took place in the past.

For each row, the total episodes reviewed in the applicable time period are shown in column 608, the number of episodes that were correctly classified are shown in the column 610, and the success percentage is shown in column 612. A similar success rate report can be provided after a therapy training session where the report may show a measurement of the agreement of therapy parameters with experts, other users, or a panel.

The screens shown in FIGS. 1-5 as well as the success rate report of FIG. 6 illustrate mere examples of how information may be gathered from and presented to the user via the user interface device. There are many alternative presentations possible. For example, the content of FIGS. 1 and 2 may be presented on a single screen. Likewise, the content of FIGS. 4 and 5 may be presented on a single screen. Many different presentation variations are possible for the training content request, the episode data, the request for the characterization data and the success rate report.

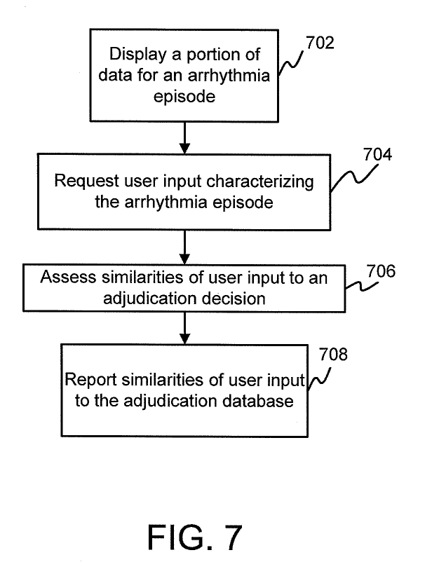

FIG. 7 illustrates the steps of one embodiment of a method for training a user. For a particular episode, the episode data or part of the episode data can be displayed to a user on a user interface at step 702. The user is asked to characterize the episode data, also via the user interface, at step 704. The characterization data could be an arrhythmia classification, therapy device parameters, or both. The characterization data also includes in some embodiments a basis for the arrhythmia classification, a rationale for the arrhythmia classification, or both. An adjudication processor processes the characterization data based on adjudication conclusions that are stored in an adjudication database at step 706. The adjudication processor may determine that the characterization data is correct, is incorrect, or determine a percentage of agreement between the characterization data and the adjudication conclusions. The level of similarities is then reported to the user at step 708.

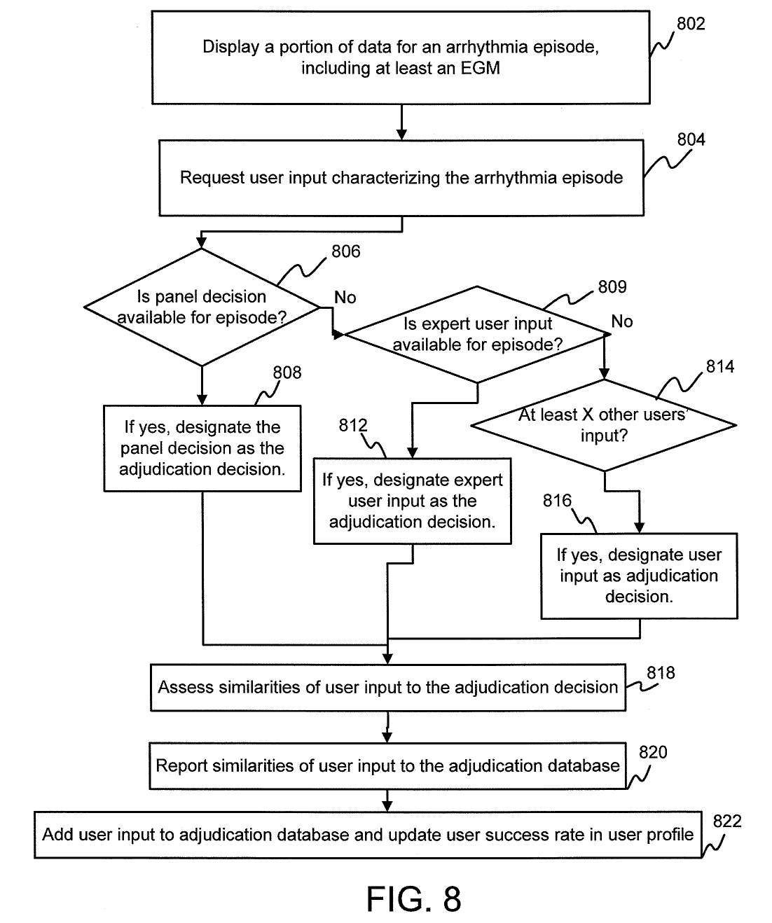

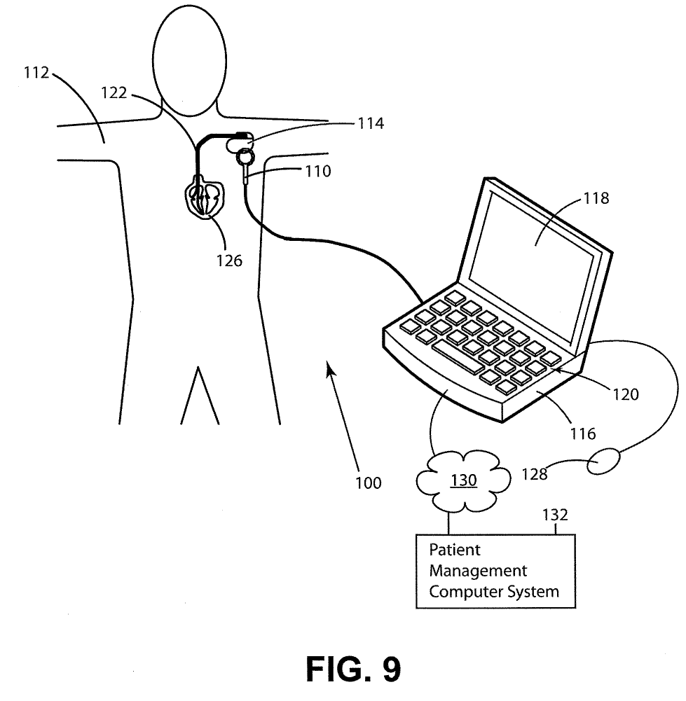

FIG. 8 illustrates the steps of a different embodiment of a method for training a user. For a particular episode, the episode data or part of the episode data can be displayed to a user on a user interface at step 802, including at least an EGM. The user is asked to characterize the episode data, also via the user interface, at step 804. The characterization data could be an arrhythmia classification, therapy device parameters, or both.

The system determines which type of adjudication decision is available for comparison to the user input. If a panel decision is available for the episode at step 806, then the panel decision is designated as the adjudication decision at step 806. If there is no panel decision, but there is an expert user or multiple expert users who have recorded input for the episode at step 809, then the expert user input is designated as the adjudication decision at step 812. If there is no expert user input, but a minimum number of other users have recorded input about the episode at step 814, then the user input is designated as the adjudication decision at step 816. The minimum number X of other users can be at least five, at least 10, at least 20, or any other threshold. If an episode does not have at least one of a panel decision, an expert user input, or a minimum number of inputs from other users, then the episode will not be presented to users during normal training sessions. Such episodes could be made available only to expert users, however, to allow input of adjudication decisions into the episode database.

At step 818, the adjudication processor processes the characterization data based on the adjudication decision. The adjudication processor may determine that the characterization data is correct, is incorrect, or determine a percentage of agreement between the characterization data and the adjudication conclusions. The level of similarities is then reported to the user at step 820. The user input is added to the adjudication database and the user success rate in the user profile is updated at step 822.

Further detailed embodiments of the hardware of the system will now be described with respect to the attached FIGS.

One embodiment of a data-generating device is a CRM device, as will now be described with reference to FIG. 9, which is a schematic of an exemplary CRM system 100. The system 100 can include an implantable medical device 114 disposed within a patient 112. The implantable medical device 114 can include pacing functionality. The implantable medical device 114 can be of various types such as, for example, a pacemaker, a cardioverter-defibrillator, a cardiac resynchronization device, a heart rhythm monitoring device, or the like. In some embodiments, the implantable medical device 114 can include one or more leads 122 disposed in or near the patient's heart 126.

The implantable medical device 114 can be in communication with an external interface system 116. In some embodiments, communication between the implantable medical device 114 and the external interface system 116 can be via inductive communication through a wand 110 held on the outside of the patient 112 near the implantable medical device 114. However, in other embodiments, communication can be carried out via radiofrequency transmission, acoustically, or the like.

The implantable medical device 114 can include one or more implantable sensors in order to gather data regarding the patient 112. For example, the implantable medical device 114 can include an activity level sensor, a respiration sensor, a blood pressure sensor, or other sensors.

The implantable medical device 114 can be configured to store data over a period of time, and periodically communicate with the external interface system 116 in order to transmit some or all of the stored data.

The external interface system 116 can be for example, a programmer, a programmer/recorder/monitor device, a computer, a patient management system, a personal digital assistant (PDA), or the like. As used herein, the term programmer refers to a device that programs implanted devices, records data from implanted devices, and allows monitoring of the implanted device. Exemplary programmer/recorder/monitor devices include the Model 3120 Programmer, available from Boston Scientific Corporation, Natick, Mass. The external interface system 116 can include a user input device, such as a keyboard 120 and/or a mouse 128. The external interface system 116 can include a video output channel and video output device, such as a video display 118 for displaying video output. The displayed video output can include a user interface screen. In addition, the video display 118 can also be equipped with a touch screen, making it into a user input device as well.

The external interface device 116 can display real-time data and/or stored data graphically, such as in charts or graphs, and textually through the user interface screen. In addition, the external interface device 116 can present a textual question to a user along with several response options. The external interface device 116 can also input and store a user's response to the question, and can store a user's text response in some embodiments.

In one embodiment, the external interface device 116, which can also be referred to as a user interface, is in communication with a patient management computer system 132. The communication link between the user interface 116 and the patient management computer system 132 may be via phone lines, the Internet 130, or any other data connection. The user interface 116 can also be used when it is not in communication with device, but is only in communication with the patient management computer system 132.

In one embodiment, the external interface device 116 is capable of changing the operational parameters of the implantable medical device 114, and is therefore referred to as a programmer. Typically, programmers are used to interface with CRM devices in a clinic or hospital setting. In this context, the user of the external interface device is a physician or trained technician.

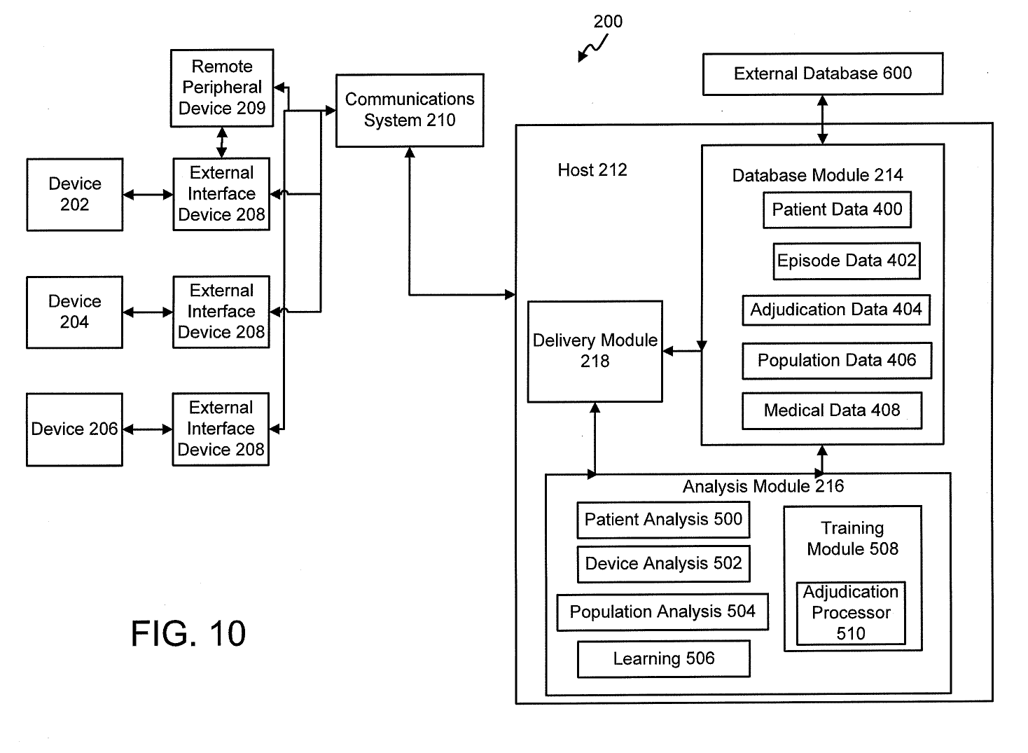

FIG. 10 is a schematic illustration of a patient management system consistent with at least one embodiment of the invention. The patient management system is capable of generating an episode database and supporting a training module. Patient management system 200 generally includes one or more devices 202, 204, and 206, one or more external interface devices 208, a communication system 210, one or more remote peripheral devices 209, and a host 212.

Each component of the patient management system 200 can communicate using the communication system 210. Some components may also communicate directly with one another. The various components of the example patient management system 200 illustrated herein are described below.

Data-generating devices 202, 204, and 206 can be implantable devices or external devices that may provide one or more of the following functions with respect to a patient: (1) sensing, (2) data analysis, and (3) therapy. For example, in one embodiment, devices 202, 204, and 206 are either implanted or external devices used to measure a variety of physiological, subjective, and environmental conditions of a patient using electrical, mechanical, and/or chemical means. The devices 202, 204, and 206 can be configured to automatically gather data or can require manual intervention by the patient. The devices 202, 204, and 206 can be configured to store data related to the physiological and/or subjective measurements and/or transmit the data to the communication system 210 using a variety of methods, described in detail below. Although three devices 202, 204, and 206 are illustrated in the example embodiment shown, many more devices can be coupled to the patient management system. In one embodiment, each of the devices 202, 204 and 206 is serving a different patient. In one embodiment, two or more devices are serving a single patient.

The devices 202, 204, and 206 can be configured to analyze the measured data and act upon the analyzed data. For example, the devices 202, 204, and 206 can be configured to modify therapy or provide alarm based on the analysis of the data.

In one embodiment, devices 202, 204, and 206 provide therapy. Therapy can be provided automatically or in response to an external communication. Devices 202, 204, and 206 are programmable in that the characteristics of their sensing, therapy (e.g., duration and interval), or communication can be altered by communication between the devices 202, 204, and 206 and other components of the patient management system 200. Devices 202, 204, and 206 can also perform self-checks or be interrogated by the communication system 210 to verify that the devices are functioning properly. Examples of different embodiments of the devices 202, 204, and 206 are provided herein.

Devices implanted within the body have the ability to sense and communicate as well as to provide therapy. Implantable devices can provide direct measurement of characteristics of the body, including, without limitation, electrical cardiac activity (e.g., a pacemaker, cardiac resynchronization management device, defibrillator, etc.), physical motion, temperature, heart rate, activity, blood pressure, breathing patterns, ejection fractions, blood viscosity, blood chemistry, blood glucose levels, and other patient-specific clinical physiological parameters, while minimizing the need for patient compliance. Derived measurements can also be determined from the implantable device sensors (e.g., a sleep sensor, functional capacity indicator, autonomic tone indicator, sleep quality indicator, cough indicator, anxiety indicator, and cardiovascular wellness indicator for calculating a quality of life indicator quantifying a patient's overall health and well-being).

Devices 202, 204, and 206 can also be external devices, or devices that are not implanted in the human body, that are used to measure physiological data (e.g., a thermometer, sphygmomanometer, or external devices used to measure blood characteristics, body weight, physical strength, mental acuity, diet, heart characteristics, and relative geographic position).

The patient management system 200 may also include one or more remote peripheral devices 209 (e.g., cellular telephones, pagers, PDA devices, facsimiles, remote computers, printers, video and/or audio devices) that use wired or wireless technologies to communicate with the communication system 210 and/or the host 212.

The example database module 214 includes a patient database 400, an episode database 402, an adjudication database 404, a population database 406, and a medical database 408, all of which are described further below. The patient database 400 includes patient specific data, including data acquired by the devices 202, 204, and 206, as well as a patient's medical records and historical information.

The population database 406 includes non-patient specific data, such as data relating to other patients and population trends. The example medical database 408 includes clinical data relating to the treatment of diseases, such as historical trend data for multiple patients in the form of a record of progression of their disease(s) along with markers of key events.

The episode database 402 has episode data regarding a plurality of different arrhythmia episodes generated from those of devices 202, 204, and 206 that provide arrhythmia episode data. The adjudication database 404 includes adjudication conclusions associated with the episode data such as arrhythmia episodes. The adjudication database 404 and the episode database 402 can actually be a single database with shared data that is used as either episode data or adjudication data depending on the particular data set being presented to the user.

Information can also be provided from an external source, such as external database 600. For example, the external database 600 includes external medical records maintained by a third party, such as drug prescription records maintained by a pharmacy, providing information regarding the type of drugs that have been prescribed for a patient or, in another example, authorization data from patient groups that have authorized users to view arrhythmia episode data.

The example analysis module 216 includes a patient analysis module 500, device analysis module 502, population analysis module 504, a learning module 506, and a training module 508. Patient analysis module 500 may utilize information collected by the patient management system 200, as well as information for other relevant sources, to analyze data related to a patient and provide timely and predictive assessments of the patient's well-being. Device analysis module 502 analyzes data from the devices 202, 204, and 206 and external interface devices 208 to predict and determine device issues or failures. Population analysis module 504 uses the data collected in the database module 214 to manage the health of a population. Learning module 506 analyzes the data provided from the various information sources, including the data collected by the patient system 200 and external information sources, and may be implemented via a neural network (or equivalent) system to perform, for example, probabilistic calculations.

Training module 508 analyzes data provided from the episode database 402, the adjudication database 404, and other portions of the patient management system 200 to determine what particular portion of episode data for one of the arrhythmia episodes from the episode database should be displayed. Training module 508 can, through the delivery module 218 described below, provide the means for graphically displaying a portion of data selected from arrhythmia episode data related to an arrhythmia episode of a patient, such as data generated by a data-generating device and stored in the episode database.

Training module 508 also determines what type of characterization data is required in response to the episode data, and can articulate the request for user input characterizing the request. The request may be a direct question to a user, a series of choices provided to the user, or simply a blank space on the user interface configured to accommodate the user input. Training module 508 can include an adjudication processor 510 that processes the characterization data relative to the adjudication database 410. Processing the characterization data can include assessing and reporting similarities of user input relative to the adjudication decision. The training module may also store the training history for individual users.

Delivery module 218 coordinates the delivery of feedback based on the analysis performed by the host 212. For example, based on the data collected from the devices and analyzed by the host 212, the delivery module 218 can deliver information to the caregiver, user, or to the patient using, for example, a display provided on the external interface device 208. The external interface device 208 is also configured, according to multiple embodiments, to display a particular portion of episode data for one episode from the episode database and receive characterization data from the user characterizing the displayed episode data for the training system. Displayed data, as described above, can be determined by the training module 508 and delivery module 218.

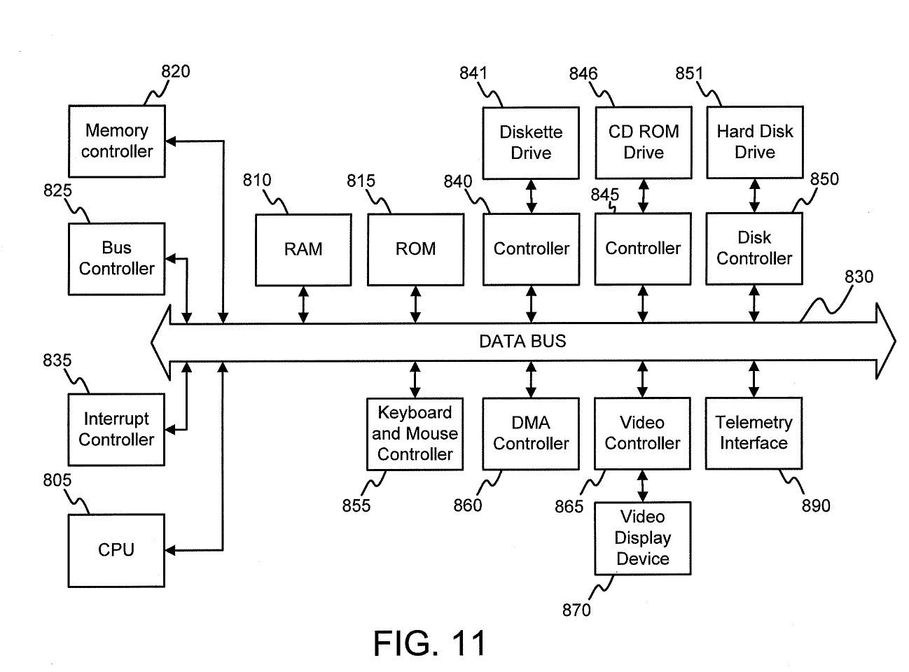

External interface devices to display information, such as programmer/recorder/monitors, can include components common to many computing devices. Referring now to FIG. 11, a diagram of various components is shown in accordance with some embodiments. However, it is not required that an external interface device have every component illustrated in FIG. 11.

In one embodiment, the external interface device includes a central processing unit (CPU) 805 or processor, which may include a conventional microprocessor, random access memory (RAM) 810 for temporary storage of information, and read only memory (ROM) 815 for permanent storage of information. A memory controller 820 is provided for controlling system RAM 810. A bus controller 825 is provided for controlling data bus 830, and an interrupt controller 835 is used for receiving and processing various interrupt signals from the other system components.

Mass storage can be provided by diskette drive 841, which is connected to bus 830 by controller 840, CD-ROM drive 846, which is connected to bus 830 by controller 845, and hard disk drive 851, which is connected to bus 830 by controller 850. User input to the programmer system may be provided by a number of devices. For example, a keyboard and mouse can connected to bus 830 by keyboard and mouse controller 855. DMA controller 860 is provided for performing direct memory access to system RAM 810. A visual display is generated by a video controller 865 or video output, which controls video display 870. The external system can also include a telemetry interface 890 or telemetry circuit which allows the external system to interface and exchange data with an implantable medical device. It will be appreciated that some embodiments may lack various elements illustrated in FIG. 11.

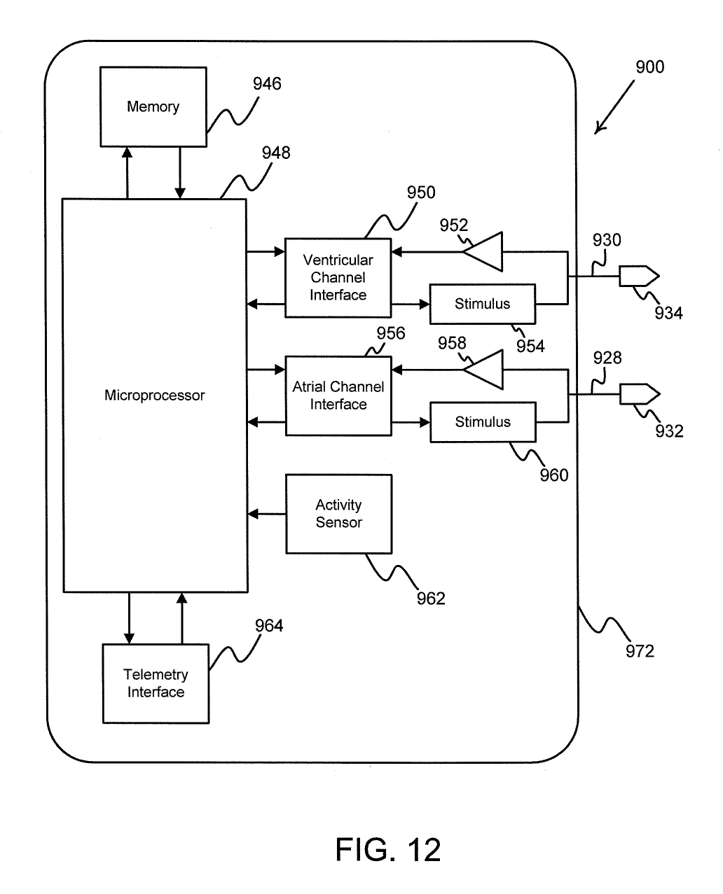

Referring now to FIG. 12, some components of an exemplary implantable system 900 are schematically illustrated. The implantable medical system 900 can include an implantable medical device 972 coupled to one or more stimulation leads 930 and 928. The implantable device 972 can also include other sensors such as activity sensor 962.

The implantable device can include a microprocessor 948 (or processor) that communicates with a memory 946 via a bidirectional data bus. The memory 946 typically comprises ROM or RAM for program storage and RAM for data storage. The implantable device can be configured to execute various operations such as processing of signals and execution of methods as described herein. A telemetry interface 964 is also provided for communicating with an external unit, such as a programmer device or a patient management system.

The implantable device can include ventricular sensing and pacing channels comprising sensing amplifier 952, output circuit 954, and a ventricular channel interface 950 which communicates bidirectionally with a port of microprocessor 948. The ventricular sensing and pacing channel can be in communication with stimulation lead 930 and electrode 934. The implantable device can include atrial sensing and pacing channels comprising sensing amplifier 958, output circuit 960, and an atrial channel interface 956 which communicates bidirectionally with a port of microprocessor 948. The atrial sensing and pacing channel can be in communication with stimulation lead 928 and electrode 932. For each channel, the same lead and electrode can be used for both sensing and pacing. The channel interfaces 950 and 956 can include analog-to-digital converters for digitizing sensing signal inputs from the sensing amplifiers and registers which can be written to by the microprocessor in order to output pacing pulses, change the pacing pulse amplitude, and adjust the gain and threshold values for the sensing amplifiers.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

One of ordinary skill in the art will understand that the modules, circuitry, and methods shown and described herein with regard to various embodiments of the invention can be implemented using software, hardware, and combinations of software and hardware. As such, the illustrated and/or described modules and circuitry are intended to encompass software implementations, hardware implementations, and software and hardware implementations.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A system for presenting arrhythmia episode information to a user, comprising:
an episode database having episode data regarding a plurality of different arrhythmia episodes generated from a plurality of data-generating devices;
a user interface configured to display at least a particular portion of episode data for one of the arrhythmia episodes from the episode database and receive characterization data from the user characterizing the displayed episode data;
an adjudication database having adjudication conclusions associated with the arrhythmia episodes in the episode database; and
an adjudication processor, configured to process characterization data relative to the adjudication database.

2. The system of claim 1 wherein the displayed episode data includes a device-generated arrhythmia classification.

3. The system of claim 2 wherein the data-generating device is a cardiac rhythm management device that is implanted in a patient, where the displayed episode data includes at least a portion of an intracardiac electrogram and a device category into which the data-generating device associated with the episode data is classified.

4. The system of claim 3 wherein the displayed episode data further includes at least one selected from the group consisting of pace and sense markers on the intracardiac electrogram, device diagnostics, the therapy delivered during the arrhythmia episode and a device-generated arrhythmia classification.

5. The system of claim 4 wherein the device-generated arrhythmia classification is one of the group consisting of nontreated tachy arrhythmia, treated arrhythmia, atrial fibrillation (AF), and supraventricular tachycardia (SVT).

6. The system of claim 1 where the characterization data includes an arrhythmia episode classification.

7. The system of claim 6 wherein the arrhythmia episode classification is selected from the group consisting of: polymorphic ventricular tachycardia (PVT), ventricular fibrillation (VF), monomorphic ventricular tachycardia (MVT), supraventricular tachycardia (SVT), AV nodal re-entrant tachycardia (AVNRT), atrial fibrillation (AF), atrial flutter (AFL), atrial tachy, sinus tachy (ST), dual tachy, unknown, pacemaker-mediated tachycardia (PMT), oversensing and noise.

8. The system of claim 6 wherein the characterization data includes a designation of which part of the episode data was the basis for the arrhythmia episode classification.

9. The system of claim 6 wherein the characterization data includes a designation of a rationale for the selection of the arrhythmia episode classification.

10. The system of claim 1 where the characterization data includes at least one therapy selection.

11. The system of claim 10 where the at least one therapy selection is chosen from the group of turning detection enhancements on or off, turning morphology-based discrimination on or off, and selecting therapy programming to be shock energy or anti-tachycardia pacing.

12. The system of claim 1 wherein at least a portion of the adjudication conclusions in the adjudication database are determined by an expert.

13. The system of claim 1 further comprising a training database that stores user information and stores a success rate of the user, wherein a success rate is determined based on a percentage of similarity between the characterization data entered by the user and the adjudication conclusions associated with the arrhythmia episode in the adjudication database.

14. The system of claim 1 wherein the user interface is configured to allow the user to select arrhythmia episodes on the basis of the one or more of the following characteristics: patient demographics, patient identity, device type, lead types, patient disease status, patient medical history, patient medication, a device-generated arrhythmia classification, arrhythmic parameters, level of difficulty and operating mode.

15. The method comprising:
graphically displaying a portion of data selected from arrhythmia episode data related to an arrhythmia episode of a patient, wherein the arrhythmia episode data was generated by a data-generating device;
requesting user input characterizing the episode;
receiving the characterization data from the user;
assessing similarities of characterization data relative to an adjudication decision; and
reporting similarities of user input relative to the adjudication decision.

16. The method of claim 15 wherein step of graphically displaying a portion of data includes displaying a device-generated arrhythmia classification and an electrogram.

17. The method of claim 15 wherein the user input characterizing the event includes an arrhythmia classification.

18. The method of claim 15 wherein the user input characterizing the event includes at least one therapy selection, where the at least one therapy selection is chosen from the group of turning detection enhancements on or off, turning morphology-based discrimination on or off, and selecting therapy programming to be shock energy or anti-tachycardia pacing.

19. The method of claim 15 further comprising:
storing a user identity and the reported similarities in a training database,
storing a success rate of the user, wherein a success rate is determined based on a percentage of similarity between the characterization data entered by the user and the adjudication conclusions associated with the arrhythmia episode in the adjudication database.

20. A method of developing a training database for training users to identify arrhythmias, comprising the steps of:
graphically displaying a portion of data selected from arrhythmia episode data;
requesting user input characterizing the event;
assessing similarities of user input relative to an adjudication decision in an adjudication database;
reporting similarities of user input relative to the adjudication decision; and
if the user is designated as a expert, recording the user input in the adjudication database.
